(19) United States
(12) Patent Application Publication
(10) Pub. No.: US 2004/0186573 A1
(43) Pub. Date: Sep. 23, 2004

(54) Annulus fibrosis augmentation methods and apparatus

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/787,469

(22) Filed: Feb. 26, 2004

Related U.S. Application Data
(63) Continuation-in-part of application No. 10/120,763, filed on Apr. 11, 2002.
Application No. 10/120,763 is a continuation-in-part of application No. 09/807,820, filed on Apr. 19, 2001.
Application No. 10/120,763 is a continuation-in-part of application No. 09/638,241, filed on Aug. 14, 2000.
Application No. 10/120,763 is a continuation-in-part of application No. 09/454,908, filed on Dec. 3, 1999, now Pat. No. 6,491,724.
Application No. 10/120,763 is a continuation-in-part of application No. 09/639,309, filed on Aug. 14, 2000, now Pat. No. 6,419,702.
Application No. 10/120,763 is a continuation-in-part of application No. 09/690,536, filed on Oct. 16, 2000, now Pat. No. 6,371,990.
Application No. 09/690,536 is a continuation-in-part of application No. 09/638,726, filed on Aug. 14, 2000, now Pat. No. 6,340,369.
Application No. 09/690,536 is a continuation-in-part of application No. 09/415,382, filed on Oct. 8, 1999, now Pat. No. 6,419,704.

(51) Int. Cl. A61F 2/44

(52) U.S. Cl. 623/17.11

(57) ABSTRACT

Annulus fibrosis reconstruction methods use devices similar to those used in reconstruction of the ACL of the knee. Tendon (allograft or autograft) or tendon connected to bones (allograft or autograft) is grafted across the disc space and into the vertebrae. The grafts can be held in position with interference screws. The screws could be resorbable. The reconstructed annulus would a) help to control the movement between the vertebrae, for example, rotation, translocation, and bending; and b) help prevent the extrusion of devices or material from the disc space. A shape-changing material, clamp or cable tie may also be used to increase the height of an intradiscal device according to the invention. Additional fixation features may be added around a hoop or band to enhance surface area or prevent “pull-out.”

### REFERENCE TO RELATED APPLICATIONS

This application is a continuation-in-part of U.S. patent application Ser. No. 10/120,763, filed Apr. 11, 2002, which is a continuation-in-part of U.S. patent application Ser. Nos.:

Ser. No. 09/807,820, filed Apr. 19, 2001, which is a 371 of PCT/US00/14708, filed May 30, 2000;

Ser. No. 09/638,241, filed Aug. 14, 2000;

Ser. No. 09/454,908, filed Dec. 3, 1999, now U.S. Pat. No. 6,491,724;

Ser. No. 09/639,309, filed Aug. 14, 2000, now U.S. Pat. No. 6,419,702;

Ser. No. 09/690,536, filed Oct. 16, 2000, now U.S. Pat. No. 6,371,990, which is a continuation-in-part of U.S. patent application Ser. Nos. 09/638,726, filed Aug. 14, 2000, now U.S. Pat. No. 6,340,369; and Ser. No. 09/415,382, filed Oct. 8, 1999, now U.S. Pat. No. 6,419,704.

The entire content of each application and patent is incorporated herein by reference.

### FIELD OF THE INVENTION

This invention relates generally to human spinal surgery and, in particular, to methods and apparatus for augmenting the annulus fibrosis while controlling vertebral motion, thereby preventing additional annular tears and attendant discomfort.

### BACKGROUND OF THE INVENTION

According to human anatomy, spinal function is dependent upon the intervertebral disc and the facet joints. In a sense, the annulus fibrosis, nucleus pulpous, and the facet joints form the legs of a three-legged stool.

To restore disc height resulting, for example, from degenerative disease, prosthetic discs are used to replace only the nucleus pulpous. Reference is made to my U.S. Pat. No. 6,419,704, which discusses spinal anatomy, spinal physiology, disc degeneration, surgical and non-surgical treatments of disc disease, and the advantages of prosthetic disc replacement.

The annulus is formed of 10 to 60 fibrous bands which serve to control vertebral motion. One half of the bands tighten to check motion when the vertebra above or below the disc are turned in either direction. Restoring disc height returns tension to the annular noted in the prosthetic disc patent application. In addition, restoring annular tension decreases annular protrusion into the spinal canal or neural foramen. Thus, decreasing annular protrusion may eliminate pressure on the spinal cord or nerve roots.

At times the rotational, translational, and axial compression forces exceed the strength of the annular fibers. The excessive forces tear the annular fibers. A single event can tear one band to all the bands. Subsequent tears can connect to previous tears of a few bands resulting in a hole through the entire annulus fibrosis. Holes through the entire annulus fibrosis can result in extrusion of the nucleus pulpous. Extrusion of the nucleus pulpous is referred to as a “herniated disc.” Disc herniation can result in back pan, neck pain, arm pain, leg pain, nerve or spinal cord injury, or a combination of the above.

Since the annulus is innervated with pain fibers, acute annular tears without herniation of the nucleus can be painful. Unfortunately, the annular tears often do not heal completely. The chronic tears can result in neck pain, back pain, shoulder pain, buttock pain, or thigh pain. The chronic tears weaken the annulus fibrosis predisposing the disc to herniation or additional annular tears. My U.S. Pat. No. 6,340,369, entitled “Treating Degenerative Disc Disease With Harvested Disc Cells and Analogies of the Extracellular Matrix,” and U.S. Pat. No. 6,419,704, entitled “Artificial Intervertebral Disc Replacement Method And Apparatus” describe methods and apparatus for occluding annular defects.

Prosthetic replacement of the nucleus pulpous alone risks future problems arising from annular tears. Patients may continue to complain of pain from the stresses placed onto the weakened annulus. Secondly, tears of the annulus could result in extrusion of the prosthetic nucleus. In addition, remaining nucleus pulpous could herniate through annular tears.

Some prosthetic disc designs attempt to replace nucleus and annular functions. In general, these designs attach the prosthetic disc to the vertebrae. Many of the techniques in this area attach the prosthetic disc to the end plates of the vertebrae with screws, spikes, flanges, or porous surfaces for bone ingrowth. My U.S. Pat. Nos. 6,245,107 and 6,419,704 describe methods and devices to assist the annulus in retaining remaining nucleus pulpous and a prosthetic nucleus. The entire contents of these applications are incorporated herein by reference.

The need remains, however, for a more extensive annulus augmentation technique. Failure at the disc vertebra interface could result in loosening of the prosthesis, however, and patients with loose prosthetic discs would likely require revision surgery.

### SUMMARY OF THE INVENTION

The invention reconstructs the annulus fibrosis using methods and devices similar to those used in reconstruction of the ACL of the knee. Tendon (allograft or autograft) or tendon connected to bones (allograft or autograft) is grafted across the disc space and into the vertebrae. The grafts can be held in position with interference screws. The screws could be resorbable. The reconstructed annulus would a) help to control the movement between the vertebrae, for example, rotation, translocation, and bending; and b) help prevent the extrusion of devices or material from the disc space.

A shape-changing material, clamp or cable tie may also be used to increase the height of an intradiscal device according to the invention. Additional fixation features may be added around a hoop or band to enhance surface area or prevent “pull-out.”

### BRIEF DESCRIPTION OF THE DRAWINGS

FIG. 1 is a sagittal cross-section of the spine and the novel tendon graft;

FIG. 2 is a lateral view of the spine and cross-coupled tendon grafts;

FIG. 3 is a lateral view of the a tendon graft;

FIG. 4 is a lateral view of a bone-tendon-bone graft;

FIG. 5 is a lateral view of a bone-tendon graft;

FIG. 6 is a lateral view of a bone-annulus-bone graft where the bone is from the vertebral body;

FIG. 7 is a view of the posterior aspect of the spine with the novel tendon graft;

FIG. 8A shows a lateral view of the way in which an allograft or autograft tendon may be connected to bones across a disc space and into the vertebra;

FIG. 8B shows an anterior view of the embodiment of FIG. 8A;

FIG. 9A illustrates a tendon-bone composite according to the invention;

FIG. 9B illustrates an annulus-vertebrae composite;

FIG. 10 illustrates an alternative embodiment wherein a synthetic material such as Gortex is used as opposed to tendon;

FIG. 11A illustrates the way in which a hoop can be placed into a disc for repair purposes;

FIG. 11B shows the hoop being inserted through the hole in the annulus;

FIG. 11C shows the hoop expanded within a disc space;

FIG. 12A illustrates a different embodiment wherein a bag containing nucleus or synthetic materials such as hydrogel are inserted into a previously implanted hoop;

FIG. 12B is a drawing that shows a second step associated with the procedure of FIG. 12A;

FIG. 12C shows the way in which intradiscal materials are placed into a band or hoop;

FIG. 12D shows the way in which a hoop may change space to hold intradiscal materials into position;

FIG. 12E is a drawing which shows the way in which a clamp or cable tie may be used to increase the height of an intradiscal device;

FIG. 12F shows how an intradiscal device is made taller through the tightening of a cable or clamp;

FIG. 13A is a drawing which shows additional fixation features;

FIG. 13B shows the fixation features of FIG. 13A in a tightened position;

FIG. 14 shows how a hoop or band could have ridges or grooves to prevent a suture from sliding;

FIG. 15 illustrates an intradiscal device having openings on the top and/or bottom to insert a biocompatible material;

FIG. 16A illustrates a wire frame without an outer shell;

FIG. 16B is a side-view drawing of the device of FIG. 16A; and

FIG. 17 shows the use of a lever-like tool for center-filling purposes.

### DETAILED DESCRIPTION OF THE INVENTION

This invention is broadly directed to reconstruction of the annulus fibrosis. Preferred embodiments use methods and devices similar to those used in reconstruction of the ACL (anterior cruciate ligament) of the knee. Tendon (allograft or autograft) or tendon connected to bones (allograft or autograft) is grafted across the disc space and into the vertebrae. Alternatively, synthetic materials, such as Gortex, may be used for the reconstruction. The grafts can be held in position with interference screws that may be resorbable. Appropriate grafts may be obtained from cadaver's Achilles tendon, tensions of the hamstring muscles, palmaris longus tendon, fascia, annulus fibrosis, patellar tendons, or other muscles, tendons, or ligaments of the body.

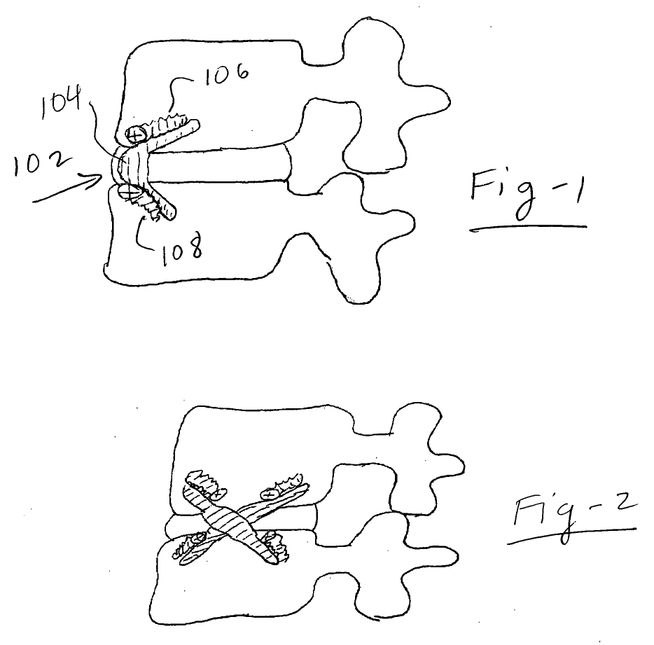

FIG. 1 is a sagittal cross-section of the spine and a tendon graft 102 according to the invention. The tendon material is shown at 104 and the screws at 106, 108. FIG. 2 is a lateral view of the spine and cross-coupled tendon grafts.

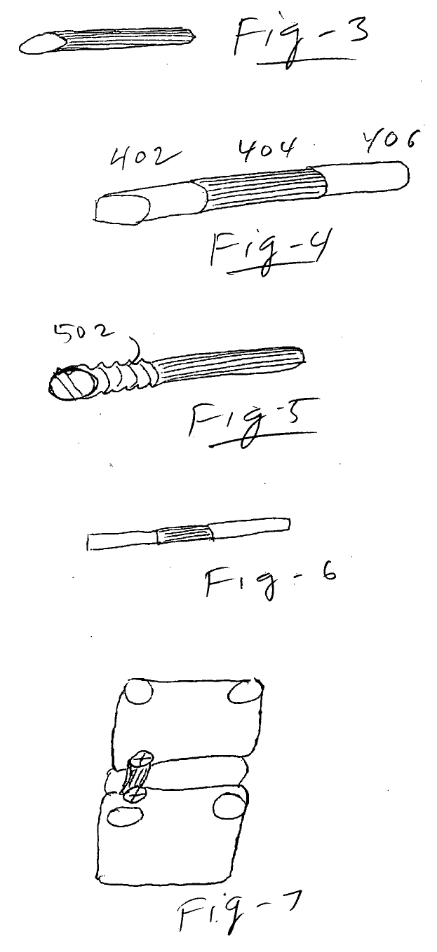

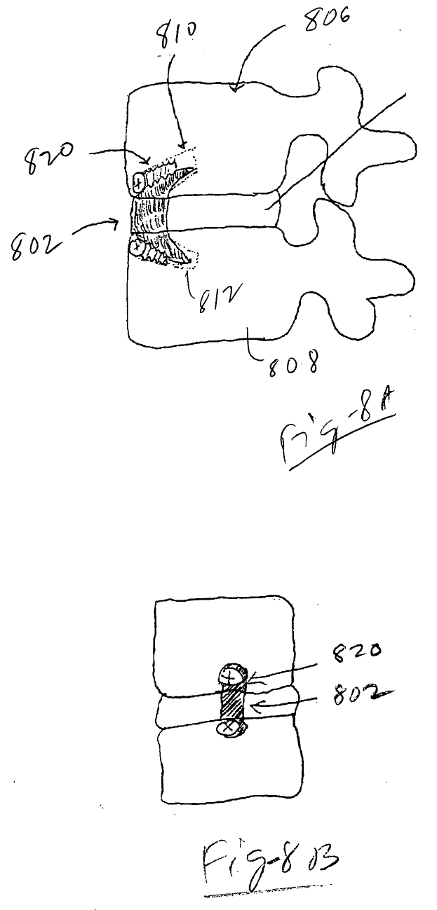

FIG. 3 is a lateral view of a tendon graft. FIG. 4 is a lateral view of a graft including bone 402, tendon 404, and bone 406. FIG. 5 is a lateral view of a bone-tendon graft having a threaded portion 502. FIG. 6 is a lateral view of a bone-annulus-bone graft where the bone portions 602, 604 are from the vertebral body. FIG. 7 is a view of the posterior aspect of the spine including a tendon graft according to the invention.

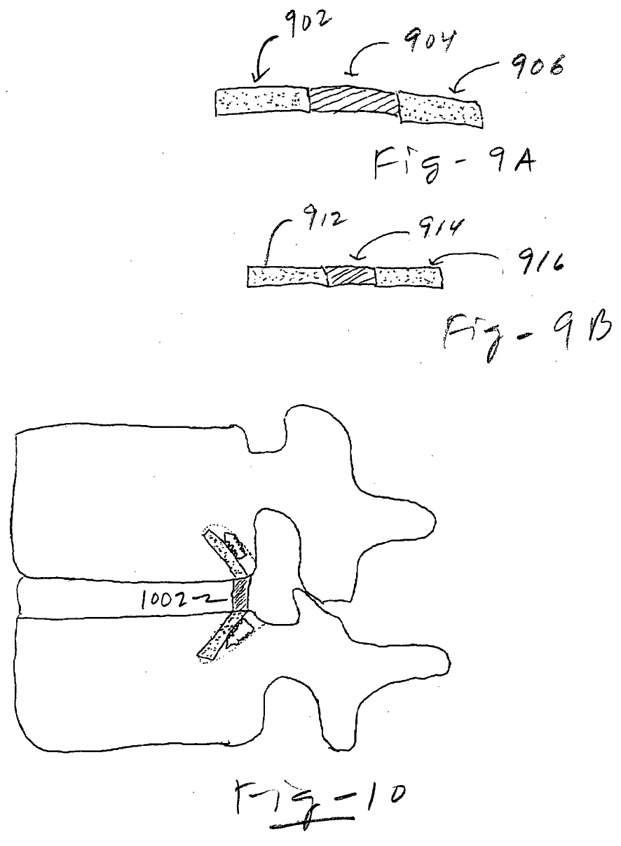

FIG. 8A shows a lateral view of the spine showing the way in which an allograft or autograft tendon 802 may be connected to bones across a disc space 804 and into the vertebra 806, 808. Procedurally, holes 810, 812 are formed in to the vertebral bodies, using drills, burrs or other conventional means, after which the tendon is inserted in either end and held in position with interference screws such as 820. FIG. 8B shows an anterior view of the embodiment of FIG. 8A.

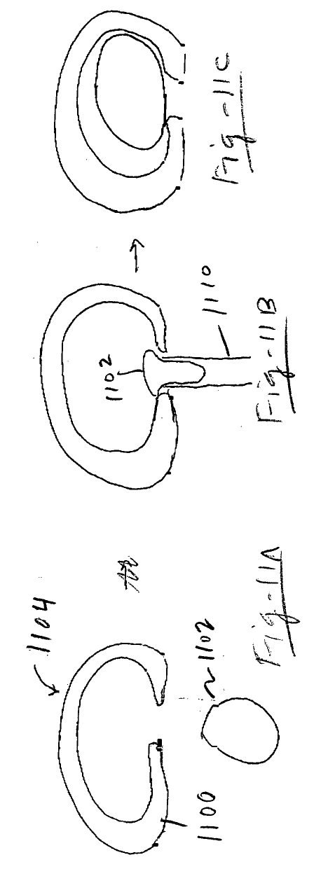

FIG. 9A illustrates a bone-tendon-bone (902-904-906) composite according to the invention. FIG. 9B illustrates a vertebrae-annulus-vertebrae (912-914-916) composite. FIG. 10 illustrates an alternative embodiment wherein a synthetic material such as Gortex 1002 is used as opposed to tendon or annulus material.

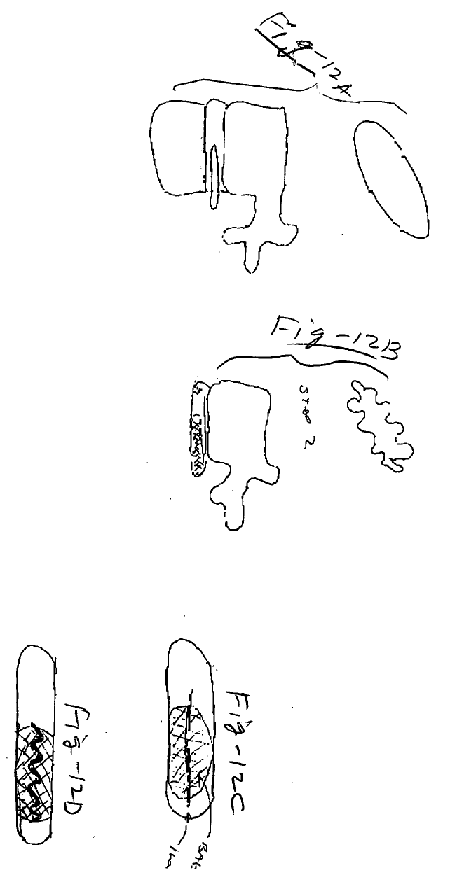

Alternative embodiments of the invention use hoops or bands to fortify the annulus. FIG. 11A illustrates the way in which a hoop 1102 can be placed into a disc 1104 through an annulus 1100 for repair purposes. FIG. 11B shows the hoop being inserted through the hole in the annulus using an inserter tool 1110. FIG. 11C shows the hoop expanded within the disc space. A shape-memory material may be useful for this purpose.

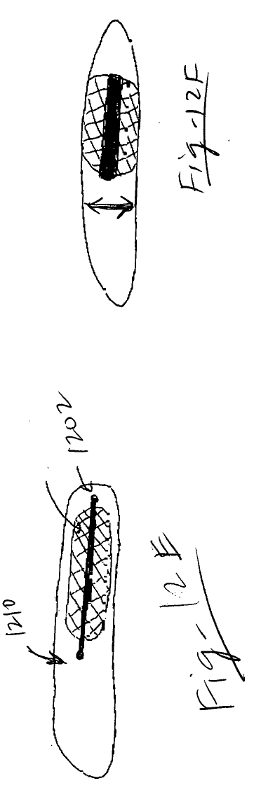

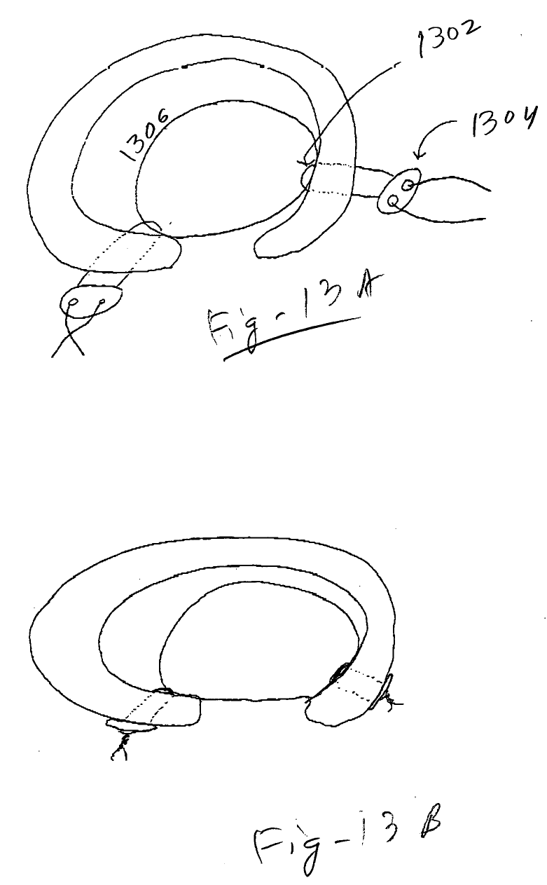

FIGS. 12A-12D illustrate a different embodiment, wherein a deformable intradiscal component is made taller using a band of material that constricts due to a shape change. In FIG. 12A, a hoop is inserted into the disc space. In FIG. 12B, an intradiscal component, such as a bag containing nucleus or synthetic materials such as hydrogel is inserted into the previously implanted hoop. In FIG. 12C, the hoop is slid over the bag, and in FIG. 12D, the hoop changes shape to hold intradiscal materials into position and/of increase the ratio of height to width of an intradiscal device.

FIG. 12E is a drawing which shows the way in which a clamp or cable tie 1202 may be used to increase the height of an intradiscal device 1204. A radiator-like clamp or a cable tie could also be used to increase the height of the bag and make the filled bag firmer. FIG. 12F shows how an intradiscal device is made taller within a disc space 1210 through the tightening of a cable or clamp.

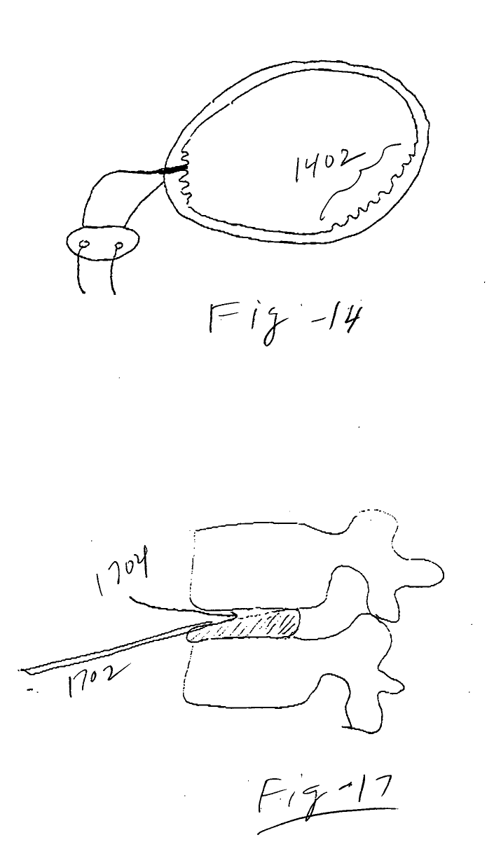

Additional fixation features such as sutures 1302 and buttons 1304 may be added around a hoop or band 1306, as shown in FIG. 13A, to enhance surface area or prevent “pull-out.” FIG. 13B shows the fixation features of FIG. 13A in a tightened position. FIG. 14 shows how a hoop or band could have ridges or grooves 1402 to prevent a suture from sliding.

FIG. 15 illustrates an intradiscal device 1500 having openings 1502, 1504 on the top and/or bottom (or a through-hole) to insert a biocompatible material 1510.

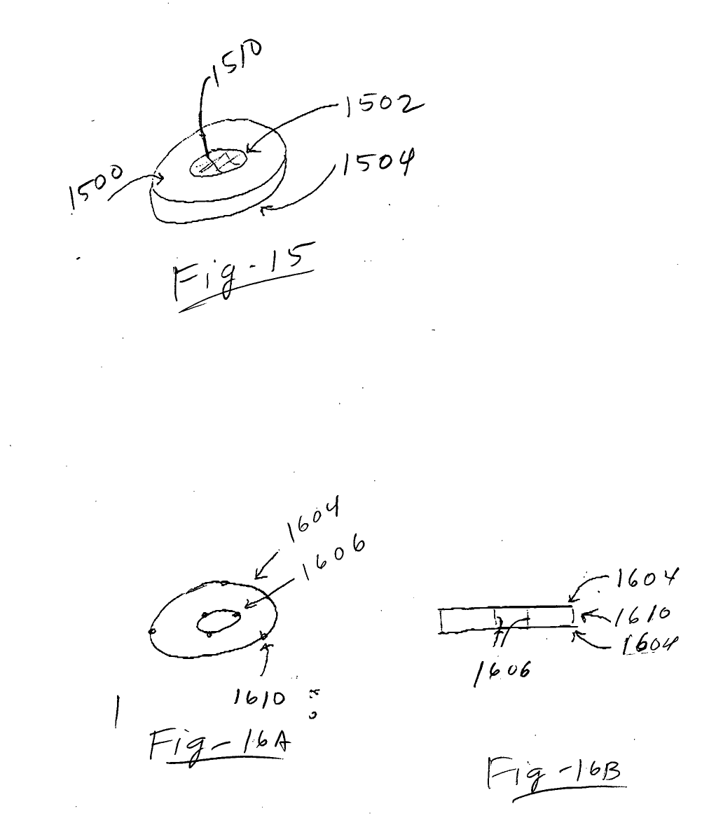

FIG. 16A illustrates the use of a wire frame comprising an outer ring 1604 and an inner ring 1606 without an outer shell. FIG. 16B is a side-view drawing of the device of FIG. 16A. Vertical supports 1610 may be used between the rings. The wire frame is collapsible to insert the device through an annular window. The frame re-expands vertically and circumferentially once in the disc space. The front of the device can be compressed to insert material after it is positioned in the disc space.

The vertical supports help to keep the bag expanded vertically, and the top and bottom contain an opening to insert material and to aid diffusion. The outer covering could be any biocompatible fabric or mesh. The inner frame would have spring-like properties but could also have other shape memory properties. FIG. 17 shows how a lever-like tool 1702 may be used to collapse the front 1704 of the device to fill the center, after which the device expands to trap the material.

I claim:

1. A method of fortifying an annulus fibrosis situated between upper and lower vertebral bodies, comprising the steps of:
providing a length of material having two ends;
forming opposing cavities in the upper and lower vertebral bodies;
inserting each end of the length of material into a respective one of the cavities in the upper and lower vertebral bodies; and
fixing the two ends of the material.

2. The method of claim 1, wherein the length of material includes an allograft or autograft tendon.

3. The method of claim 1, wherein the length of material includes a section of annulus fibrosis.

4. The method of claim 1, wherein the length of material includes Gortex or other synthetic material.

5. The method of claim 1, wherein the ends of the material are formed of allograft or autograft bone.

6. The method of claim 1, wherein the ends of the material are fixed using screws.

7. The method of claim 6, wherein the screws are bioresorbable.

8. The method of claim 1, including two lengths of material placed in criss-crossing fashion.

9. A method of at least partially occupying an intradiscal space situated between upper and lower vertebral bodies, comprising the steps of:
inserting a band of material within the intradiscal space;
placing an intradiscal device within the band; and
allowing the band to change shape, or tightening the band, to increase the height of the intradiscal device.

10. The method of claim 9, further including the steps of affixing the band of material to one or more points of an annulus fibrosis.

11. The method of claim 9, wherein the band of material is filled after being positioned within an intradiscal space.

12. The method of claim 9, wherein the intradiscal device is a filled bag.

13. The method of claim 12, wherein the bag is filled with a hydrogel.

14. The method of claim 12, wherein the bag includes a therapeutic material.
